(19) United States
(12) Patent Application Publication
(10) Pub. No.: US 2012/0089208 A1
(43) Pub. Date: Apr. 12, 2012

(54) System for Therapeutic Application of Heat

(76) Inventor: James Robert York, Frederick, MD (US)

(21) Appl. No.: 12/901,525

(22) Filed: Oct. 9, 2010

(51) Int. Cl. A61F 7/08

(52) U.S. Cl. 607/100

(57) ABSTRACT

A low profile, mobile system for therapeutic application of heat such as far infrared (“FIR”) heat to an affected area of an individual's body including straps and a pad, the pad including a plurality of FIR emitting discs and heaters. The FIR emitting discs are ceramic discs of cordierite made of a mixture of magnesium oxide, aluminum oxide, silicon oxide and/or trace elements. The heat application system is designed for simple operation via a single button.

### BACKGROUND OF THE INVENTION

Embodiments of the present invention generally relate to systems for therapeutic application of heat. More specifically, the present invention relates to systems and methods for therapeutic application of heat such as far infrared heat to areas of the body such as the lower back region.

Therapeutic application of heat to various locations on an individual's body is commonly employed to relieve muscle tension and to increase blood flow to injured tissues to promote healing thereof. An individual typically applies therapeutic heat to his or her body via a heating pad or other apparatus that generates heat.

The heat applied to the individual's body from a heating pad may be generated by placing hot water inside a heating pad or microwaving a fluid-filled heating pad to heat the fluid contained therein. It is also commonly known to generate heat via electric heating pads. Electric heating pads employ electric energy to generate thermal energy for application to an individual's body.

Additionally, some disposable heating pads typically generate heat via exothermic reactions. In some instances, the reaction begins when the heating pad is exposed to the air. In other applications, the user must begin the exothermic reaction by mixing two reactants. For example, a heating pad typically includes a reservoir holding a first reactant and a pellet internal to said same reservoir containing a second reactant. A user typically activates the heating pad by breaking the pellet contained within the reservoir to release the second reactant and allow it to mix with the first reactant, upon which the exothermic reaction begins and heat is generated.

Additionally, a therapeutic application of heat may include application of far infrared heat energy to an individual's body. In some instances, far infrared heat may be generated by lamps using light emitting diode arrays. To benefit from the far infrared heat, a patient must sit in a position in which the heat reaches the effected portion of the body. Typically, such far infrared heat generating lamps must receive electrical power and are not mobile.

Therapeutic far infrared heat may also be generated via treatment of a mat or layer of a material such as fiberglass or polyester film with a substance possessing FIR emitting properties. More specifically, the layer of material is treated with high and low resistance carbon. When heated by a heater such as a coiled electric heater, the carbon treated fibers emit FIR heat.

### BRIEF SUMMARY OF THE INVENTION

In one aspect of the present invention, a system for therapeutic application of heat to an affected area of an individual's body is provided. The system includes: straps; and a pad, the pad coupled to the straps, the pad including: at least one temperature sensor; and a plurality of heat generating apparatus, each of the heat generating apparatus including a heater and an FIR emitting disc, the FIR emitting disc being a ceramic disc of cordierite; wherein a temperature of the FIR emitting discs is controlled via control of the heaters; and wherein the control of the heaters is controlled based upon a temperature of the FIR emitting discs as sensed by the at least one temperature sensor.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a rear perspective view of a system for therapeutic application of heat in accordance with one embodiment of the present invention wherein the straps are coupled to each other;

FIG. 2A is a front view of the system of FIG. 1 wherein the straps are uncoupled and extended throughout the same plane as the pad;

FIG. 2B is a rear view of the system of FIG. 1 wherein the straps are uncoupled and extended throughout the same plane as the pad;

FIG. 3 is a cross-sectional view of the pad of the system of FIG. 1 taken along lines 3-3 of FIG. 2A;

FIG. 4 is a rear schematic view of the internal components of the pad of the system of FIG. 1 in accordance with one embodiment of the present invention;

FIG. 5A is a schematic view of the internal components of the user interface unit of the system of FIG. 1 in accordance with one embodiment of the present invention; and

FIGS. 6A and 6B depict a flowchart of the steps of a process to control the functions of the system of FIG. 1 in accordance with one embodiment of the present invention,

### DETAILED DESCRIPTION OF THE INVENTION

Certain terminology may be used in the following description for convenience only and is not limiting. The words “lower” and “upper” and “top” and “bottom” designate directions in the drawings to which reference is made. The terminology includes the words above specifically mentioned, derivatives thereof and words of similar import.

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a disc” may include a plurality of discs. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs and materials are now described. All publications mentioned herein are incorporated herein by reference in their entirety. Where there are discrepancies in terms and definitions used in references that are incorporated by reference, the terms used in this application shall have the definitions given herein.

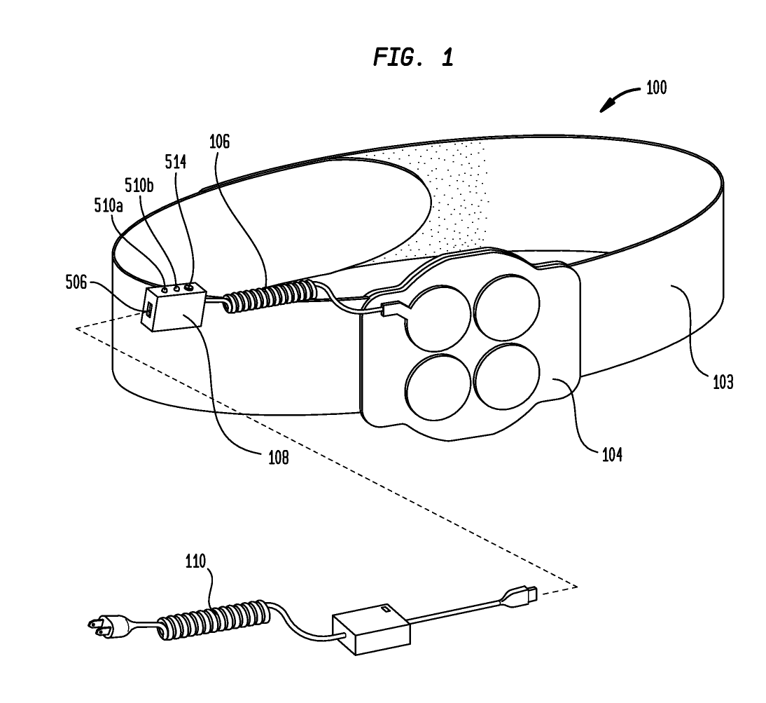

Referring first to FIG. 1, depicted is a rear perspective view of heat application system 100 in accordance with one embodiment of the present invention. Heat application system 100 includes, inter alia, fastening mechanism 102, pad 104, cabling 106, user interface unit 108, and power supply charger 110. Heat application system 100 is designed to be a low profile, mobile back support for therapeutic application of heat such as FIR heat to an affected area of an individual's body. Heat application system 100 is also designed for simple operation via a single button. Furthermore, the design of the pad and belt of heat application system 100 provides targeted, deep penetrating heat to the user's lower back region and other large muscle groups.

In the embodiment of the invention described in the present application, heat application system 100 is designed for application of heat (e.g., far infrared (“FIR”) heat) to a user's middle and/or lower back. To accomplish this, heat application system 100 is removably attached to the body of a patient via coupling of fastening mechanism 102 to allow heat to be applied to the back as further described below. However, the system of the present invention may also be easily modified to accommodate application of heat to areas of the body other than the middle and/or lower back including, but not limited to, the upper back, neck, shoulder, elbow, wrist, knee, ankle, hip, and other large muscle groups.

Additionally, alternate embodiments of the present invention are envisioned in which fastening mechanism 102 and/or pad 104 are provided in a different form to accommodate application of heat to areas of the body other than the middle and/or lower back. For example, straps such as straps 103 may be worn over the shoulder and around the torso to accommodate application of heat to the scapula, low neck and shoulder region. In addition, the straps and pad of the present invention may be easily modified to allow the pad to cover the hip and other large muscle groups (e.g., quadriceps) for application of heat thereto.

Further, alternate embodiments of the present invention are envisioned in which fastening mechanism 102 and/or pad 104 are modified to accommodate application of heat to the body of an animal such as a horse or a dog. For example, heat application system 100 may be easily modified to allow it to be worn around the torso of such an animal to provide targeted heat to the back and hind legs of the animal. The complete portability of heat application system 100 allows it to be utilized for such purpose.

Furthermore, alternative embodiments of the present invention are envisioned in which the apparatus employed in the present invention is adapted for incorporation in survival suits. That is, a plurality of heat generating assemblies as described herein may be incorporated into a survival suit in strategic locations to raise the core body temperature of the individual. Such an embodiment may be employed, for example, to raise the core body temperature of an individual suffering from hypothermia or to prevent the core body temperature from dropping when an individual is in extremely cold conditions.

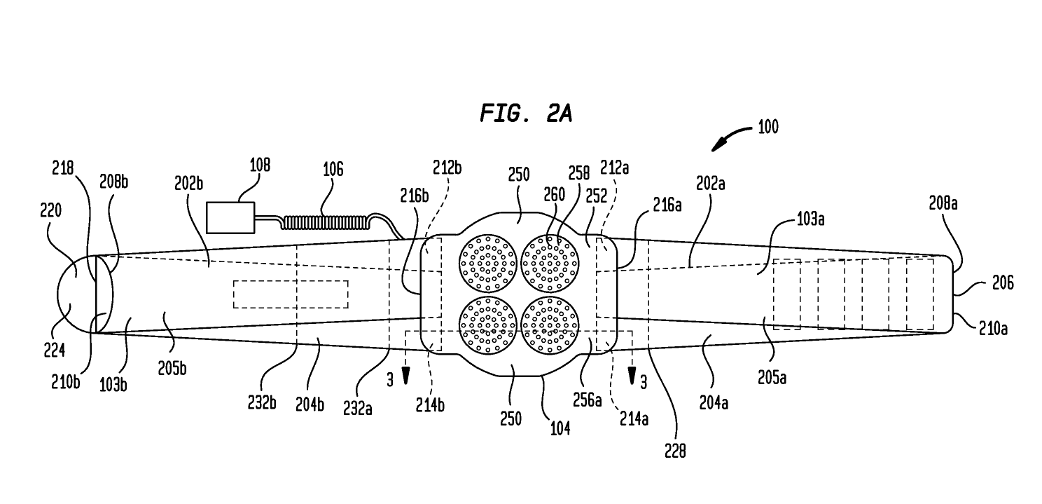

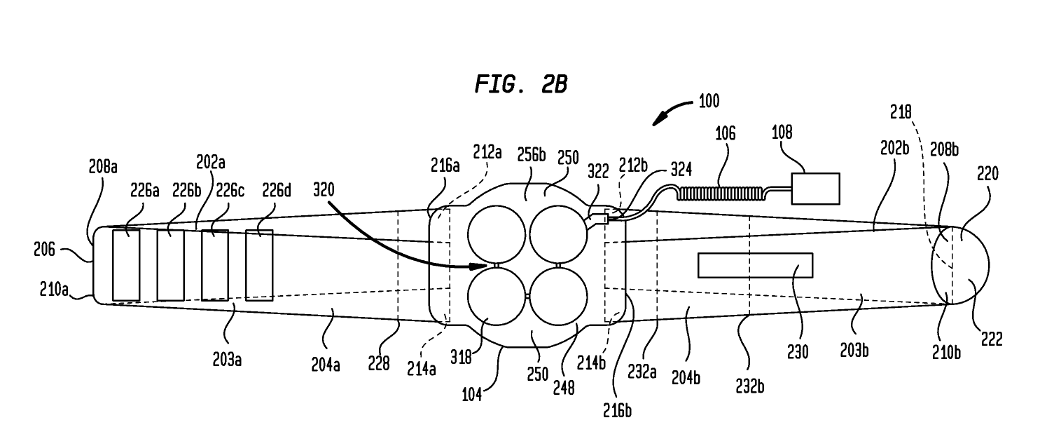

Turning now to FIGS. 2A and 2B, depicted are front and rear views of heat application system 100, respectively. In the embodiment of the present invention depicted in FIG. 2A, fastening mechanism 102 includes straps 103a and 103b. Strap 103a is located to the right of pad 104 and it includes front strip 202a and rear strip 204a. Front and rear strips 202a and 204a, respectively, may be manufactured of any suitable fabric including, but not limited to, an elastomeric fabric that is 80% nylon and 20% spandex. However, any fabric possessing the appropriate elastic properties may be employed without departing from the scope of the present invention.

In the depicted embodiment of the present invention, front and rear strips 202a and 204a, respectively, include smooth rearward facing surfaces 203a and forward facing surfaces 205a, the latter of which include the “loop” portion of a hook and loop closure system throughout the entire surface area thereof.

Front and rear strips 202a and 204a, respectively, are substantially rectangular in shape with substantially equal heights and lengths. The proximal, latitudinal edge 212a of front strip 202a is coupled to the topmost portion of right latitudinal edge 216a of pad 104, and the proximal, latitudinal edge 214a of rear strip 204a is coupled to the bottommost portion of right latitudinal edge 216a of pad 104. Front and rear strips 202a and 204a, respectively, are coupled at downwardly and upwardly angles, respectively, of approximately 22.5 degrees relative to top and bottom edges, respectively, of latitudinal edge 216a of pad 104. This angling of front and rear strips 202a and 204a, respectively, allows the distal latitudinal ends 208a and 210a, respectively, thereof to be coupled together to form distal edge 206. In the depicted embodiment, distal end 208a of front strip 202a and distal end 210a of rear strip 204a are sewn together to form edge 206. However, front strip 202a and rear strip 204a may be coupled to each other via alternative attachment methods without departing from the scope of the present invention.

As best seen in FIG. 2B, strap 103a also includes a plurality of substantially rectangular fastening mechanisms 226a-226d. Fastening mechanism 226a is located at the distal end of strap 103a and is approximately the same height as front and rear strips 202a and 204a, respectively. Fastening mechanisms 226b-226d are substantially the same height and are configured in an approximately equally spaced, linear arrangement along the distal end of strap 103a as depicted in FIG. 2b. Spacing is provided between each fastening mechanism 226 to allow a patient greater flexibility in coupling heat application system 100 to his or her torso as discussed in greater detail below. In the depicted embodiment, four (4) fastening mechanisms 226 are included. However, greater or lesser quantities of fastening mechanisms may be substituted without departing from the scope of the present invention.

Each fastening mechanism 226 is a sheet of the “hook” portion of a hook and loop fastening system that is non-removably attached vertically across both front strip 202a and rear strip 204a. That is, front strip 202a, rear strip 204a, and fastening mechanism 226 are stitched together around the periphery of each fastening mechanism 226. This stitching provides additional stability to strap 103a via a sturdy coupling of all three layers at each fastening mechanism 226. Specifically, fastening mechanisms 226 minimize the potential of front strip 202a and rear strip 204a stretching unevenly when heat application system 100 is coupled to the torso a patient. In other words, as a patient extends strap 103a to encircle his or her torso, the portion of front strip 202a and rear strip 204a located longitudinally outward from each fastening mechanism 226 will be prevented from stretching.

In addition to fastening mechanisms 226a, strap 103a is also reinforced by substantially vertical stitching 228. Stitching 228 provides additional stability of strap 103a via coupling of front strip 202a to rear strip 204a in a substantially vertical manner that extends throughout the height of strap 103a.

As best seen in the front view of FIG. 2A, strap 103b is located to the left of pad 104 and it includes elastomeric front strip 202b and elastomeric rear strip 204b. In the depicted embodiment of the present invention, front and rear strips 202b and 204b are manufactured of an identical or similar material as front and rear strips 202a and 204a, respectively, as discussed in detail above. Additionally, in the depicted embodiment of the present invention, front and rear strips 202b and 204b, respectively, include smooth rearward facing surfaces 203b and forward facing surfaces 205b, the latter of which include the “loop” portion of a hook and loop closure system throughout the entire surface area thereof.

Front and rear strips 202b and 204b, respectively, are substantially rectangular in shape with substantially equal heights and lengths. The proximal, latitudinal edge 212b of front strip 202b is coupled to the topmost portion of left latitudinal edge 216b of pad 104, and the proximal, latitudinal edge of rear strip 204b is coupled to the bottommost portion of left latitudinal edge 216b of pad 104. Front and rear strips 202b and 204b, respectively, are coupled at downwardly and upwardly angles, respectively, of approximately 22.5 degrees relative to top and bottom edges, respectively, of left latitudinal edge 216b of pad 104. This angling of front and rear strips 202b and 204b, respectively, allows the distal latitudinal ends 208b and 210b, respectively, thereof to be coupled together in combination with closure mechanism 220 along vertical intersection 218. That is, in the depicted embodiment, distal end 208b of front strip 202b and distal end 210b of rear strip 204b are sewn together with proximal edge 218 of closure mechanism 220 along vertical intersection 218. However, front strip 202b and rear strip 204b may be coupled to each other via alternative attachment methods without departing from the scope of the present invention.

Substantially semi-circular closure mechanism 220 is located at the distal end of strap 103b. It includes front layer 224 and rear layer 222. Front layer 224 is a semi-circular sheet of the loop portion of a “hook and loop” fastening system. Rear layer 222 is a sheet of rubber and is included to provide additional strength to closure mechanism 220. Front layer 224 and rear layer 222 are sewn together around their entire perimeter. Additionally, distal end 210b of rear strip 204b and distal end 208b of front strip 202b extend between front layer 224 and rear layer 222 and are non-removably coupled therebetween when front layer 224 and rear 222 are sewn together. In this manner, closure mechanism 220 is securely coupled to the distal end of strap 103b.

In addition, strap 103b includes substantially rectangular stabilizing strip 230 on its rear surface. Strip 230 is sewn to front strip 202b and rear strip 204b around the periphery of strip 230. Stabilizing strip 230 is substantially perpendicular to latitudinal edge 216a and is horizontal aligned with an approximate midpoint of latitudinal edge 216a. Stabilizing strip 230 is provided to serve a similar stabilizing function as fastening mechanisms 226 as discussed in further detail above with respect to strap 103a. However, stabilizing strip 230 is cut from the same fabric as front strip 202 and rear strip 204. That is, stabilizing strip 230 possesses the same elastic properties as the other components of strap 103b. Therefore, when a patient extends strap 103b, the entire length of strap 103b may stretch. In this manner, additional stability is provided to strap 103b without compromising the extendibility thereof.

Strap 103b also includes vertical stitching 232a and 232b, which is provided for additional stability of strap 103b. Stitching 232 couples front strip 202b and rear strip 204b and extends throughout the height of strap 103b. Stitching 232a is located at a distance from left longitudinal edge 216b that is approximately equal to the distance between stitching 228 and right longitudinal edge 216a. Stitching 232b is located at the approximate horizontal midpoint of stabilizing strip 230.

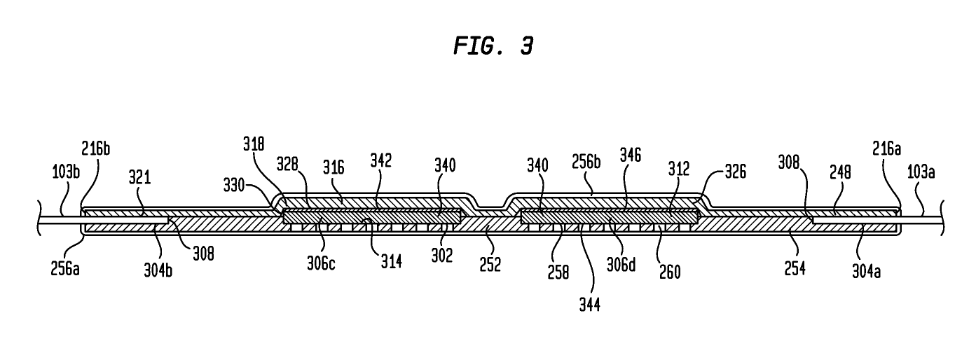

Still referring to FIGS. 2A and 2B, pad 104 is located between straps 103a and 103b. Pad 104 is substantially rectangular in shape with the exception of two substantially identical arc-shaped flanges 250 extending from the top and bottom thereof. As best seen in FIG. 3, pad 104 includes two layers of foam (i.e., pad front 252 and pad rear 248) stitched together around their periphery with the exception of the area for the opening 324 of conduit 322. In the depicted embodiment, the foam is ethyl-vinyl acetate (“EVA”), however, other similar materials may be substituted without departing from the scope of the present invention.

Still referring to FIG. 3, pad front 252 has a substantially flat outwardly facing surface 254 with a layer of fabric 256a or a similar material irremovably adhered thereto via an adhesive or the like. Fabric 256a is 82% nylon and 18% spandex and is provided for visual and tactile aesthetic purposes. Additionally, outwardly facing surface 254 is substantially planar with the exception of four (4) substantially circular heat openings 258. Each heat opening 258 includes a plurality of perforations 260 that facilitate transference of the heat generated by heat application system 100 to the back of a patient.

Pad rear 248 has an outwardly facing surface 316 with a layer of fabric 256b or a similar material irremovably adhered thereto via an adhesive or the like. Fabric 256b is 82% nylon and 18% spandex and is provided for visual and tactile aesthetic purposes. Outwardly facing surface 316 is substantially planar with the exception of four (4) substantially circular heat generating apparatus protrusions 318, a plurality of channels 320 (FIG. 2B), and conduit 322 (FIG. 2B). As best seen in FIG. 2B, heat generating apparatus holders 318 are located in substantially the same position as the four (4) heat openings 258 of pad front 252 (the latter of which are best seen in FIG. 2A). Channels 320 are provided to allow electrical connection between each heat generating apparatus. More specifically, in the depicted embodiment, three (3) channels 320 are configured to allow electrical wires to pass therethrough and connect the electrical components within pad 104 to user interface unit 108 via cabling 106 as described in greater detail below. Conduit 322 includes conduit opening 324 and is provided to allow cabling 106 to pass therethrough as discussed in greater detail below.

Referring now to the cross-sectional view of FIG. 3, pad front 252 has an inwardly facing surface 302 that is substantially planar with the exception of substantially rectangular strap recessions 304a and 304b and heat generating apparatus recesses 306a-306d.

Strap recessions 304 extend inward from latitudinal edges 216 to points 308. Additionally, strap recessions 304 extend throughout the entire height of edge 216 and are provided for encasement of the proximal ends of straps 103a and 103b. That is, a portion of the proximal end of strap 103 is placed in strap recession 304 and is non-removably attached to pad 104 via the stitching of the periphery of pad front 252 and the periphery of pad rear 248 to proximal end of strap 103.

Heat generating apparatus recesses 306 are substantially circular in shape and are provided for retention of the forwardmost portions of heat generating apparatus 312. Additionally, the innermost ends of perforations 260 pass through inwardly facing surfaces 314 of recesses 306 and the remaining portion of perforations 260 pass through pad front 252. In this manner, heat emitted by heat generating apparatus 312 passes through perforations 260 to the back of a patient, the latter of which is located adjacent the outwardly facing surface of pad front 252 when heat application system 100 is worn by the patient. While all four (4) recesses 306a-306d are not visible in the cross-sectional view of FIG. 3, their arrangement substantially corresponds to the arrangement of the four (4) heat openings 258 depicted in FIG. 2A.

Pad rear 248 also has an inwardly facing surface 321 that is substantially planar with the exception of four (4) substantially circular heat generating apparatus holders 326, channels 320 (FIG. 2B), and conduit 322 (FIG. 2B). Each heat generating apparatus holder 326 includes a substantially planar and circular inwardly facing surface 328 and holder walls 330, the latter of which proceed downward until they intersect inwardly facing surface 321. Channels 320 and conduit 322 (not shown in cross-section) extend upward from inwardly facing surface 321 at a height approximately equal to the height of heat generating apparatus holder 326. Channels 320 form passages for interconnecting wires for the heat generating apparatus and conduit 322 forms a passageway through which cabling 106 passes.

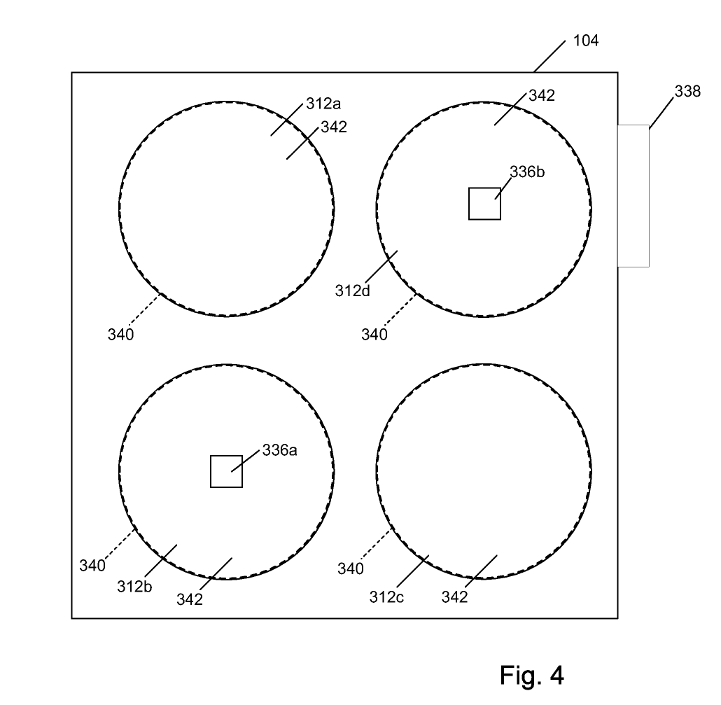

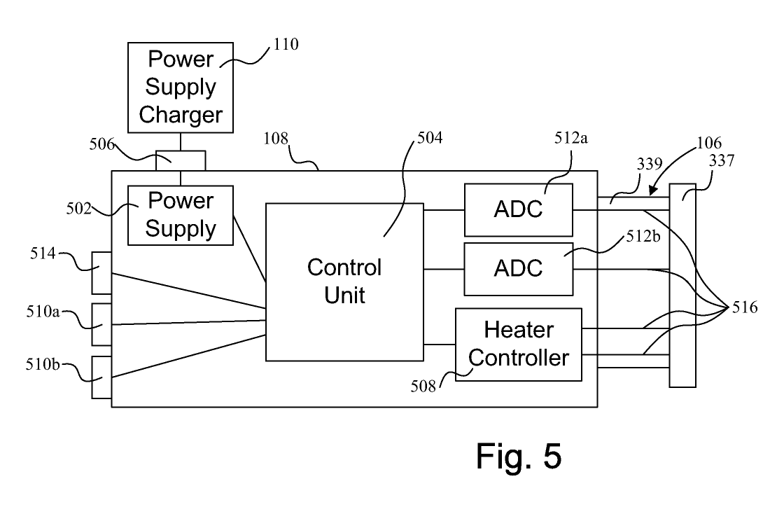

Referring now to FIG. 4, depicted is a block diagram of the internal components of pad 104 in accordance with the present invention. In this embodiment, pad 104 includes, inter alia, four (4) heat generating apparatus 312a-312d, two (2) temperature sensors 336, and connector 338. In the depicted embodiment of the present invention, temperature sensors 336 are analog temperature sensors that sense the temperature at heat generating apparatus 312b and 312d, respectively, and convert it to an analog voltage that may be read by control unit 504 (See FIG. 5). Temperature sensors 336 may be virtually any temperature sensing component that is compatible with the other components of pad 104 including, but not limited to, a Microchip MCP9701A Linear Active Thermistor Integrated Circuit. Although the depicted embodiment of the present invention includes two (2) temperature sensors, an alternate quantity may be substituted without departing from the scope of the present invention.

Connector 338 is the female portion of a push-pull type connector, and it is provided to couple pad 104 to user interface unit 108 via cabling 106. In the depicted embodiment, connector 338 has six pins; however, varying quantities of contacts may be substituted without departing from the scope of the present invention. In the depicted embodiment, connector 338 is model no. SLK-M608XXYY as manufactured by Sunshine Electronics Co., Ltd. However, other connectors compatible with the other components of heat application system 100 may be substituted. As discussed in greater detail below, connector 338 couples to a corresponding male portion 337 of a push-pull type connector, the latter of which is nonremovably coupled to an end of cabling 106.

Pad 104 also includes four (4) heat generating apparatus 312. In the depicted embodiment, each heat generating apparatus 312 includes an FIR emitting disc 340 and a heater 342. FIR emitting discs 340 are shown as dashed lines as they are substantially identical in size to heaters 342, and they are located directly in front of same. In the depicted embodiment, FIR emitting discs 340 have a thickness of approximately three (3) mm. FIR emitting disc 340 is a Cordierite ceramic with a mixture of 45-55% silicon oxide (“SiO₂”), 30-40% magnesium oxide (“MgO”), 10-20% aluminum oxide (“Al₂O₃”), and, potentially, trace elements. In the depicted embodiment of the present invention, the FIR emitting disc is fired at a temperature of 2300 degrees Fahrenheit to achieve optimal hardening of FIR emitting disc 340. However, disc 340 may be fired at other temperatures without departing from the scope of the present invention. The composition of FIR emitting disc 340 provides optimum therapeutic application of heat through the desired temperature range of approximately 100 degrees through 110 degrees. That is, although FIR emitting disc 340 emits electromagnetic radiation through a range of 3 μm to 16 μm, when FIR emitting disc 340 is heated to the desired temperature range of 103 to 108 degrees, FIR emitting discs 340 emit electromagnetic radiation through a range of 9 μm to 12 μm. It has been found that the energy emitted by FIR emitting discs 340 is greatest in the range of 9 μm to 12 μm, and is therefore most beneficial for therapeutic purposes. That is, in this range, therapeutic FIR heat is most effective in penetrating body tissues and being absorbed thereby. After penetration into body tissues, FIR heat transforms from light energy into thermal energy. Thermal energy within the tissues promotes dilation of blood vessels and capillaries to promote healing and circulation and to relax lower back muscles for greater muscle flexibility and relief. It also increases blood flow, oxygenation, and circulation, which promotes healing of body tissues. Enhanced penetration of FIR heat, therefore, allows FIR emitting disc 340 be highly effective for therapeutic application of heat, particularly when heated to a temperature in the desired temperature range.

FIR emitting discs 340 are substantially cylindrical in shape with substantially planar forward facing surface 344 and substantially planar rearward facing surface 346. Forward facing surface 344 contacts inwardly facing surface 314 of each recess 306 of pad front 252 as described in greater detail above. Rearward facing surface 346 of each FIR emitting disc 340 is nonremovably coupled to a respective one of heaters 342 via an adhesive or the like.

The composition of FIR emitting discs 340 also allows each disc to be relatively thin while still emitting FIR heat in the range of 9 μm-12 μm in the desired temperature range and resisting damage due to bending, cracking, or the like. As best seen in FIG. 3, the overall thickness of pad 104 is relatively thin. In the depicted embodiment, the overall thickness of pad 104 is approximately one half inch and the width of pad 104 is approximately ten inches. The relatively thin thickness of pad 104 provides a heat application system 100 that is more comfortable for the wearer as it does not protrude from a user's back to a degree that would be uncomfortable or inconvenient for that individual. That is, when heat application system 100 is coupled around the torso of an individual as discussed above, the thickness of pad 104 is thin enough that it does not encumber movement of the individual nor prevent him or her from use of a chair or other form of seating. An individual wearing heat application system 100 should have the ability to raise and lower himself or herself normally with relative comfort in virtually any commonly known seating apparatus.

In the depicted embodiment, heaters 342 are flexible etched foil silicon heaters of dimensions compatible with the other components of heat application system 100. Silicon flex heaters are as manufactured by Watlow Electric Manufacturing Company. However, the shape and dimensions of heaters 342 are custom made for incorporation in the heat application system 100 of the present invention. That is, heaters 342 are customized to be substantially circular in shape with a diameter of approximately 2 15/32 inches (i.e., 61 mm) and a thickness of 0.018 inches. Silicon flex heaters are included due to their relatively thin profile in order to minimize the overall profile and thickness of pad 104 to maximize comfort for the wearer of heat application system 100 as discussed above. The silicon flex heaters also provide increased structure that acts to protect a wearer in the event one or more FIR emitting discs 340 chip or fracture. That is, the adhesive that binds heater 342 to its respective FIR emitting disc 340 will retain any chipped or fractured pieces adjacent 342 and will minimize the potential that such pieces fall from, or relocate within, heat application system 100 in a manner in which they may harm a user. Furthermore, silicon flex heaters assist in directing the heat generated by FIR emitting discs 340 by blocking the heat from traveling in a direction opposite of the body. In other words, silicon flex heaters impede the heat from traveling away from the body, which maximizes the quantity of heat transferred to the body. However, heaters other than silicon flex heaters including, but not limited to, coiled wire heaters or metal strip heaters may be substituted without departing from the scope of the present invention.

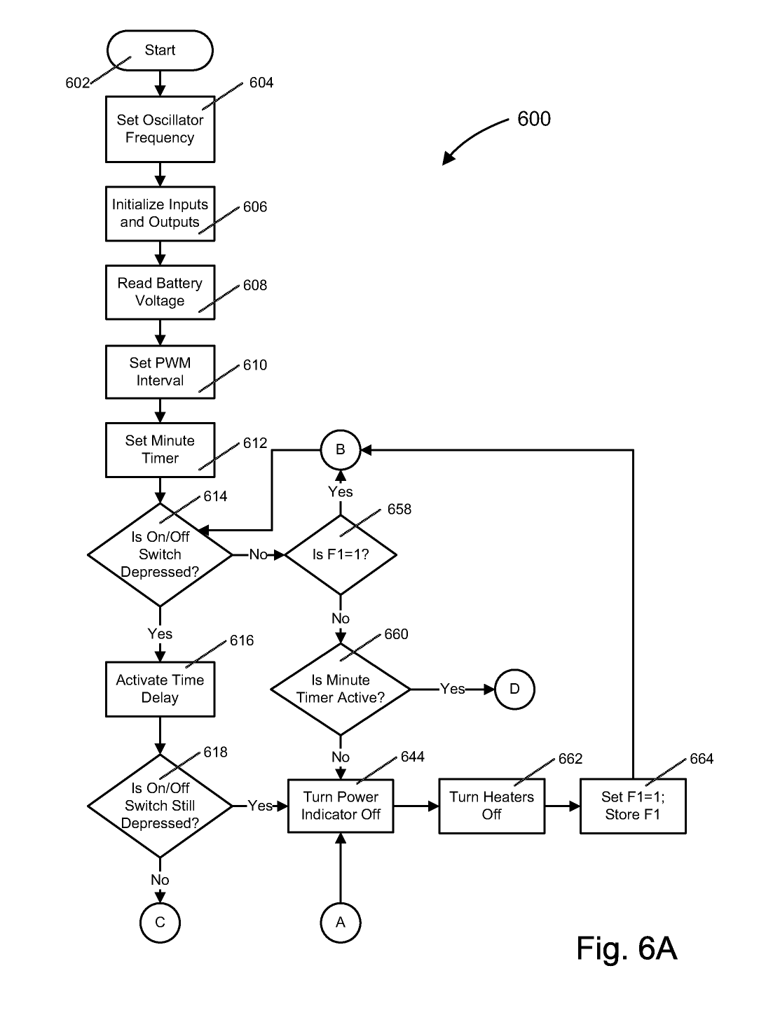

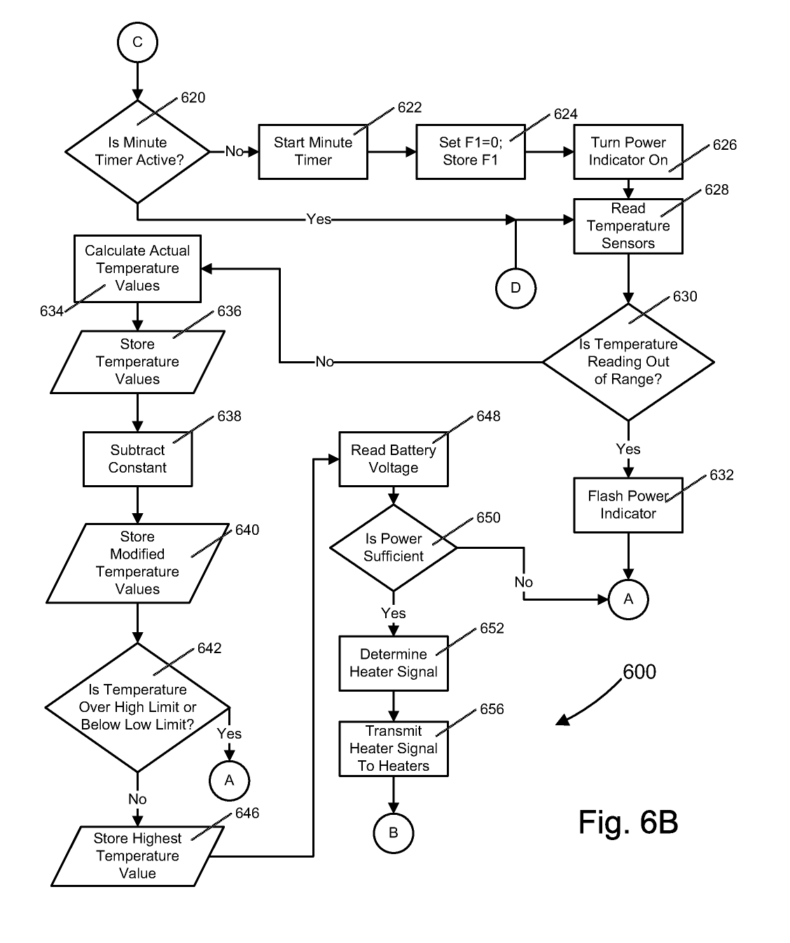

Also, as depicted in FIG. 4, heaters 342 have substantially the same diameter as FIR emitting discs 340. As discussed in greater detail below with respect to FIG. 6, electric energy provided to heaters 342 by user interface unit 108 through cabling 106 is converted to thermal energy by heaters 342. The thermal energy is transferred to FIR emitting discs 340, thereby gradually increasing the temperature thereof until FIR emitting discs 340 reach a temperature in the desired range of approximately 103 to 108 degrees. Once the temperature enters this range, it is controlled to maintain a temperature of 107.6 degrees Fahrenheit (42 degrees Celsius) as discussed below in greater detail relative to process 600 of FIG. 6. As discussed above, in this temperature range, FIR heat is emitted in an optimal therapeutic range of 9 μm-12 μm.

Turning now to FIG. 5, depicted is a block diagram of one embodiment of the internal components of user interface unit 108. User interface unit 108 includes, inter alia, cabling 106, connector 337, power supply 502, control unit 504, charger connector 506, heater controller 508, indicators 510, ADC 512a, ADC 512b, and switch 514.

In the depicted embodiment, power supply 502 includes a two-cell lithium polymer battery with a nominal voltage of 7.4 volts and a maximum discharge of 1.8 amperes. Power supply 502 is electrically connected to control unit 504 and the other components of user control interface 108 and provides electrical power thereto. However, power supply 502 may be any power supply that is compatible with and capable of supplying power to the other components of user control interface 108, including but not limited to lithium ion, nickel-metal hydride, and alkaline batteries.

Power supply charger 110 is provided for charging of power supply 502. Power supply charger 110 may be any suitable charger that is compatible with power supply 502 and capable of charging same. Power supply charger 110 may derive its power from a household receptacle, personal computer, automobile, or the like to facilitate a variety of locations for charging and/or use of heat application system 100. In the depicted embodiment, power supply charger 110 is model no. FSKD 1200800U as manufacturer by Universal Power Group. However, any suitable charger may be substituted without departing from the scope of the present invention.

Power supply charger connector 506 is located in an external surface of user interface unit 108. In the depicted embodiment, it is the female portion of a universal serial bus (USB) mini-B type connector. Power supply charger connector 506 is provided to receive the male portion of USB mini-B type connector, the latter of which is included with a power supply charger such as power supply charger 110. That is, power supply charger connector 506 provides an interface for the transference of electrical power from power supply charger 110 to power supply 502 for storage therein as is commonly known in the art.

Control unit 504 of user interface 108 is programmed with software such as that depicted in FIGS. 6A through 6B to control the functions of heat application system 100. That is, control unit 504 may be programmed with an algorithm capable of, for example, reading temperature sensors 336 and controlling the power provided to heaters 342. More specifically, control unit 504, inter alia, executes software that senses the temperature at heat generating apparatus 340 via temperature sensors 336 to determine if more or less power should be provided to heaters 342. In addition, the software executed by control unit 504 performs other functions such as timing and controlling the duration that power is provided to heaters 342 as described in greater detail below with respect to FIG. 6.

In the depicted embodiment, control unit 502 is a digital signal processor such as model no. PIC16F616 as manufactured by Universal Power Group, however, the present invention is not so limited. Any combination of hardware and software capable of executing a process such as that depicted in FIGS. 6A-6B may be substituted for any component described herein without departing from the scope of the present invention.

Heater controller 508 controls heaters 336 by outputting a specific electrical signal to heaters 336a to 336d as determined by process 600 as described in greater detail below with respect to FIG. 6. In the depicted embodiment, heater controller 508 is a Pulse Width Modulation (“PWC”) controller having model no. 86342 as manufactured by Universal Power Group. However, alternate heater controllers may be substituted without departing from the scope of the present invention. Or, heater controller may be integral to control unit 504. Heater controller 508 is electrically connected to control unit 504 and is controlled thereby. Additionally, heater controller 508 is electrically connected to heaters 336 via cabling 106 and connector 337, the latter of which plugs into connector 338 of pad 104. In the depicted embodiment of the present invention, all four (4) heaters 342 are wired together to function as a single heater. However, alternate embodiments of the present invention are envisioned in which one or more heaters may be individually controlled.

User interface unit 108 also includes indicators 510 to notify the user of the current status of heat application system 100. In the depicted embodiment of the present invention, indicators 510 include one or more light emitting diodes (“LEDs”). The LED(s) are electrically connected to control unit 504 and are controlled thereby as described in greater detail below with regards to FIG. 6.

More specifically, in the depicted embodiment of the present invention, indicator 510a includes red and green LEDs that illuminate to indicate the status of power supply charger 110. That is, the red LED of indicator 510a is energized when power supply charger 110 is increasing the electrical charge stored in power supply 502. When power supply 502 has stored the maximum amount of electrical energy, the green LED lamp is illuminated to signal to the user that power supply 502 is fully charged.

User interface unit 108 also includes indicator 510b, which indicates various statuses associated with heat application system 100. In the depicted embodiment, indicator 510b includes one or more green LEDs. These LED(s) are illuminated in a steady manner when heat application system 100 is turned on by the user to indicate that system 100 is powered on. Indicator 510b also flashes when either temperature sensor reading is out of range. The functions of indicator 510b are described in greater detail below with respect to FIGS. 6A and 6B and process 600. Although indicators 510a and 510b utilize LEDs, other forms of indicators may be substituted without departing from the scope of the present invention.

User interface unit 510 also includes analog to digital converters (“ADC”) ADC 512a and ADC 512b. These ADCs convert the analog input signals received from sensors 336 into a digital value that may be read by control unit 504. The digital values represent the temperature values read by sensors 336. ADCs 512 are electrically connected to control unit 504, however, alternate embodiments of the present invention are envisioned in which ADCs are incorporated internal to control unit 504. ADCs are also electrically connected to temperature sensors 336 via cabling 106 and connectors 337 and 338.

Switch 514 is located in an external face of user interface unit 108 as depicted in FIG. 1. In the depicted embodiment, switch 514 is a push button switch wired to control unit 504 as a binary input. That is, when switch 514 is depressed, the status of the binary signal changes from open to closed, which is read by control unit 504 and is processed in accordance with its programmed instructions such as process 600 as described in greater detail below with respect to FIG. 6. Similarly, a user's release of switch 514 changes the status of the signal from closed to open to notify control unit 504 that switch 514 is no longer depressed. Although the depicted embodiment of the invention includes a switch, any other binary device capable of notifying control unit 504 of the action desired by the user may be substituted without departing from the scope of the present invention.

Referring now to FIGS. 1 and 5, cabling 106 is nonremovably coupled to user interface unit 108. Cabling 106 provides an electrical connection between pad 104 and user interface unit 108. Cabling 106 includes sheathing 339 and connector 337. Sheathing 339 is a pliable polymer material molded to encase electrical wires 516. For the convenience of the user, sheathing 339 is designed to cause cabling 106 to coil upon itself as depicted in FIG. 1, reducing the overall unextended length thereof.

In the depicted embodiment, connector 337 is the male portion of a push-pull type connector, and is it provided to couple cabling 106 to pad 104. Connector 337 mates with connector 338 (FIG. 4), the latter of which is the female portion of a push-pull type connector. In the depicted embodiment, connector 337 is a Sunshine Electronics Co., Ltd connector having part no. SLK-M608XXYY. Also, in the depicted embodiment, connectors 337 and 338 have six contacts to correspond with the six wires 516 of cabling 106, however, varying connectors and/or connectors with varying quantities of contacts may be substituted without departing from the scope of the present invention to accommodate differing wiring configurations.

Turning now to FIG. 6, depicted is a process 600 for controlling the functions of heat application system 100. In the depicted embodiment of the present invention, process 600 is executed by control unit 504 of user interface unit 108. Process 600 begins at step 602. Process 600 is initialized whenever power supply 502 is supplied with power (typically this power is supplied by power supply charger 110) after it has been previously depleted of all power. In other words, process 600 stops when the battery loses all power and it is re-initialized each time power supply 502 is charged to bring it out of the “dead” state. Process 600 then proceeds to 604.

At step 604, process 600 sets the oscillator frequency. In the depicted embodiment of the present invention, the oscillator frequency is set to 4 MHz as dictated by the selection and/or design of control unit 504. However, alternate frequencies may be substituted.

Process 600 then proceeds to step 606, at which the inputs and outputs are initialized. That is, at step 606, control unit 504 determines which pins are to receive inputs from the other components of heat application system 100 and which pins are to send outputs to the other components of heat application system 100. After completion of step 606, process 600 proceeds to step 608.

At step 608, process 600 reads the current voltage of power supply 504. As discussed in further detail above, in the depicted embodiment, power supply 504 is a 2-cell lithium polymer battery. Therefore, step 608 of process 600 reads the battery to determine the current charge stored therein. This information is received as an analog input to control unit 504 and it is stored in memory for later use by process 600.

Next, at step 610, process 600 sets the pulse width modulation (PWM) interval to a value of one (1) kilohertz in order to set the interval at which the pulse width modulation controller operates.

At step 612, process 600 sets the minute timer to a value of zero (0). That is, control unit 504 sets an integral timer that has been designated to function as a minute timer to a value of zero (0). A value of zero (0) causes the minute timer to be inactive until later activated by process 600. The minute timer is utilized by process 600 to track the duration for which power is supplied to heaters 342 and, therefore, the duration through which therapeutic heat is generated by heat application system 100 and presumably applied to a user's body. In the present embodiment of the invention, therapeutic heat is generated for a duration of forty-five (45) minutes as it is believed that this is the maximum time necessary to achieve optimum results. However, longer or shorter durations of therapeutic heat generation/application may be substituted without departing from the scope of the present invention. As discussed in further detail below, when the minute timer reaches forty-five (45) minutes, it becomes inactive. This inactive state causes process 600, inter alia, to remove power from heaters 342, thereby causing pad 104 to cool down in response to the cool down of its integral heat generating apparatus 312.

Next, at step 614, process 600 determines if switch 514 is depressed. In the depicted embodiment of the present invention, switch 514 is wired to a binary input and control unit 504 reads the status of the binary input (i.e., open or closed) to determine whether the switch is depressed. If control unit 504 determines that the switch is depressed, process 600 proceeds to step 616.

At step 616, a switch timer set for a duration of two (2) seconds is activated. That is, control unit 504 sets an integral timer that has been designated to function as a switch timer to a value of two (2) seconds. The switch timer is utilized by process 600 to track the duration for which switch 514 is held in a depressed state. In the present embodiment of the invention, if switch 514 is held in a depressed state for two (2) seconds, process 600 determines that the user is holding the switch in a depressed state in order to disable heat application system 100. However, longer or shorter durations may be substituted without departing from the scope of the present invention. Process 600 remains at step 616 until the switch timer has elapsed, at which point it proceeds to step 618.

At step 618, the status of switch 514 is queried via reading of the respective binary input to determine if switch 514 is still depressed. As in step 614, control unit 504 reads the status of the binary input (i.e., open or closed) to determine whether the switch is still depressed. If yes, process 600 proceeds to step 644 to execute a shutdown sequence.

At 644, control unit 504 de-illuminates the power indicator (e.g., indicator 510b as discussed above). For example, at step 644, indicator 510b may be de-illuminated by removing power from a green LED lamp. De-illumination of indicator 510b indicates to the user that heat application system 100 has been successfully de-activated by the user and generation of therapeutic heat will now cease. Process 600 then proceeds to 662.

At 662, heat generating apparatus 312 are de-activated. In the depicted embodiment, this is performed by removing the current heater signal or setting the current heater signal to zero (0) or another zero heat setting.

Next, at step 664, the value of variable F1 is set to equal one (1) and this value is stored in a memory location dedicated to variable F1 and located internal to control unit 504.

As discussed below with respect to step 658, the value of variable F1 determines the steps executed by process 600 based upon whether heat application system 100 is currently generating heat or whether it has recently completed a forty (45) minute duration of heat generation (or has otherwise been deactivated). A value of zero (0) for the variable F1 indicates that the minute timer has not yet elapsed, whereas a value of one (1) for the variable F1 indicates that heat generation system 100 has recently completed a forty-five (45) minute duration heating cycle or has otherwise been de-activated.

Alternatively, if at step 618, switch 514 is not depressed, process 600 proceeds to step 620. At step 620, process 600 determines if the minute timer is active. That is, in step 620, control unit 504 determines if the minute timer has a value other than zero (0). If no, the minute timer is not active, therapeutic heat is not currently being generated by heat application system 100, and process 600 proceeds to step 622.

At step 622, the minute timer is activated by setting it to a value of forty-five (45) minutes. Next, at step 624, the value of variable F1 is set to equal zero and this value is stored in a memory location dedicated to variable F1 and located internal to control unit 504. As discussed below with respect to step 658, the value of variable F1 determines the steps executed by process 600 based upon whether heat application system 100 is currently generating heat or whether it has completed a forty (45) minute duration of heat generation or has otherwise been deactivated.

Next, at step 626, control unit 504 illuminates the power indicator (e.g., indicator 510b as discussed above). For example, at step 626, indicator 510b is illuminated by providing power to a green LED lamp. Illumination of indicator 510b indicates to the user that heat application system 100 has been successfully activated by the user and generation of therapeutic heat will now commence. In the depicted embodiment, power may be provided to indicator 510b for the duration of the minute timer to indicate to the user that therapeutic heat is being continually generated by heat application system 100.

Next, at step 628, the values of the digital signal provided by ADC 512a and 512b are read by control unit 504. As discussed in detail above with respect to FIG. 5, ADCs 512 convert the analog value read from temperature sensors 336 to a digital value representative of the temperature at heat generating apparatus 312b and 312d.

Next, at step 630, control unit 504 determines if either of the values received from ADCs 512 are out of range. An out of range value (i.e., a temperature value that is outside a normally expected range) may be due to a loose connection (e.g., between connectors 337 and 338), failed temperature sensor 336, or other lack of signal from either sensor 336. That is, control unit 504 determines if either of the received temperature values indicates that there has been a temperature sensing failure. If a failure is detected, process 600 proceeds to step 632, at which control unit 504 causes indicator 510b to flash as an indication to the user that a temperature sensing failure has occurred. Process 600 then proceeds to step 644, at which a shutdown of heat application system 100 is initiated as described in greater detail above.

Alternatively, if step 630 determines that the temperature values are not out of range, process 600 proceeds to step 634, at which the actual temperature values are calculated from the digital input values received in step 628. In order to calculate the actual temperature at each sensor 336, control unit 504 calculates the temperature in Celsius based upon a ratio of 19.5 millivolts per degree Celsius. This value is then converted from Celsius to Fahrenheit. Process 600 then stores the temperature values calculated in step 636 in one or more internal memory locations dedicated for this purpose at step 636.

Next, at step 638, a constant of the value of 0.4 volts is subtracted from the values calculated in step 634. This value is subtracted from the temperature values in order to calibrate the temperature reading. This calibration value may be changed in order to modify the temperature range in which the system operates. That is, a smaller value for this constant directs heat application system 100 to control for a higher temperature setpoint and vice versa. Process 600 then stores the temperature values calculated in step 638 in one or more internal memory locations dedicated for this purpose at step 640.

Process 600 then executes step 642, at which it is determined if either of the values calculated in step 638 (and saved in step 640) is over the high limit or below the low limit. That is, step 638 determines if either of the modified temperature values is greater than or equal to 110 degrees Fahrenheit or less than 70 degrees Fahrenheit. If yes, process 600 proceeds to step 644, at which a shutdown of heat application system 100 is initiated as described in greater detail above. Alternatively, if both of the temperature values are in the appropriate range, process 600 stores the higher temperature value in an internal memory location designated for this purpose at step 646.

At step 648, process 600 reads the current voltage of power supply 504. As discussed in further detail above, in the depicted embodiment, power supply 504 is a 2-cell lithium polymer battery. Therefore, step 648 of process 600 determines the current charge stored therein.

Next, at step 650, process 600 determines whether there is sufficient battery power to provide power to the heaters based upon the current voltage of power supply 504. For example, in the depicted embodiment, the maximum voltage of power supply 504 is 8.8 volts. However, sufficient power may be provided to heaters 342 as long as power supply 504 has a voltage of five (5) volts or higher. Therefore, to determine whether there is sufficient battery power, the value of the minimum required voltage (i.e., five) is divided by the current voltage to determine a percentage from approximately 60 percent (i.e., when the current voltage of the battery is 8.8) to 100% (i.e., when the current voltage of the battery is 5). If the result of this calculation is greater than 100%, there is insufficient voltage in the battery (i.e., a voltage less than five (5) volts) and process 600 proceeds to 644 at which point it shuts down as described in detail above. Alternatively, if the result of this calculation is equal to or less than 100%, process 600 proceeds to step 652.

At step 652, the heater signal is calculated based upon the highest temperature value stored in step 646 above. Initially, as heaters 342 raise the temperature of heat generating apparatus 312 from ambient temperature to approximately 90 degrees Fahrenheit, the heater signal is indexed to be 100%. Once the highest temperature reaches a value of approximately 90 degrees Fahrenheit, the heater signal is reduced to 62.5%. Finally, upon reaching a highest temperature value of approximately 102 degrees Fahrenheit, the heater signal is further reduced to 12.5%. If no heat sources other than the human body are applied to heat application system 100, it is designed to maintain an appropriate temperature at pad 104 by simply maintaining a constant heater signal of 12.5%, which is of sufficient strength to replace the heat that is constantly provided, or lost, to the body of the patient and/or the outside environment. If the highest sensed temperature exceeds the desired temperature, the heater signal will be cycled between 0%, 12.5%, 62.5%, and 100% as necessary to maintain the desired temperature. It should be noted that the % signal is the portion of the heating cycle for which power is provided to the heater. The duration of the heating cycle is determined by the PWM interval as set in step 610 as discussed above.

Next, at step 656, the heater signal value is provided to heater controller 508 by control unit 504. As discussed in further detail above, heater controller 508 is a pulse width modulator that converts the heater signal value received from control unit 504 to a PWM signal and transmits the signal to heaters 336. It should be noted that heater controller 508 outputs the same PWM signal to all four (4) heaters 342. In this manner, the temperature generated by the four heat generating apparatus 312 is approximately equal to provide a relatively constant temperature across the face of pad 104 and, therefore, the back of the wearer. Transmission of the appropriate PWM signal to heat generating apparatus 312 modifies or maintains the amount of heat being generated by heat generating apparatus 312 as necessary based upon the temperatures sensed by sensors 336.

After providing a heater signal to all four heat generating apparatus 312, process 600 returns to step 614, as depicted in FIG. 6A. Step 614 again determines if switch 514 is currently depressed as discussed above. Process 600 returns to step 614 in order to determine if the user is pressing switch 514 and therefore desires to stop generation of heat by heat application system 100. If switch 514 is depressed, process 600 proceeds to 616 as discussed in detail above. If switch 514 is not depressed, process 600 proceeds to step 658.

At 658, process 600 determines if the value of variable F1 is equal to one. As discussed in further detail above with respect to step 664, if the value of variable F1 is equal to one, then the heat generating apparatus 312 have been recently deactivated due to the expiration of the minute timer, user deactivation, etc. In this scenario, process 600 returns to 614 and continually repeats step 614 and 658 until a user of heat generation system 100 activates a new heating cycle by depressing switch 514.

Alternatively, if, at 658, the value of variable F1 is not equal to one (1), then its value must be equal to zero (0). This value indicates that a current heat generation session is still underway and process 600 proceeds to step 660. At step 660, process 600 determines if the minute timer is still active. If yes, process 600 continues to step 628 and performs steps 628 through 656 to ensure that heat generation system 100 is safely operating and to adjust the output to heat generating apparatus 312 as needed to maintain the desired temperature generated by pad 104 as described in detail above. Alternatively, if at 660 the minute timer is not active, the current heat generation session should end due to expiration of the session time. Consequently, process 600 proceeds to 644 to perform the shutdown sequence described in greater detail above.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A system for therapeutic application of heat to an affected area of an individual's body comprising:
straps; and
a pad, said pad coupled to said straps, said pad including:
at least one temperature sensor; and
a plurality of heat generating apparatus, each of said heat generating apparatus including a heater and an FIR emitting disc, said FIR emitting disc being a ceramic disc of cordierite;
wherein a temperature of said FIR emitting discs is controlled via control of said heaters; and
wherein said control of said heaters is controlled based upon a temperature of said FIR emitting discs as sensed by said at least one temperature sensor.

2. A system according to claim 1, wherein said ceramic disc of cordierite is a mixture of 30-40 percent magnesium oxide, 10-20 percent aluminum oxide, and 45-55 percent silicon oxide.

3. A system according to claim 1, wherein the FIR emitting discs emits FIR heat in the range of 3-16 μm, and most beneficially in the range of 9-12 μm.

4. A system according to claim 1, wherein the FIR emitting discs are substantially cylindrical in shape and have a thickness of approximately 3 mm.

5. The system of claim 1, wherein said heaters gradually increase said temperature of said FIR emitting discs until they reach a designated temperature at which therapeutic FIR heat is emitted.

6. The system of claim 5, wherein said designated temperature is in the range of 103 to 108 degrees.
